(19) United States
(12) Patent Application Publication
(10) Pub. No.: US 2025/0152506 A1
(43) Pub. Date: May 15, 2025

(54) METHODS OF ADMINISTERING THERAPEUTIC AGENTS TO THE SUBRETINAL SPACE

(71) Applicant: EMMETROPE OPHTHALMICS LLC, Menlo Park, CA (US)

(72) Inventors: Jeffrey L. Goldberg, Menlo Park, CA (US); Noelia J. Kunzevitzky, Menlo Park, CA (US); Roger Goldberg, Menlo Park, CA (US)

(21) Appl. No.: 18/839,414

(22) PCT Filed: Feb. 17, 2023
(86) PCT No.: PCT/US2023/062786

§ 371 Date: Aug. 16, 2024

Related U.S. Application Data
(60) Provisional application No. 63/311,718, filed on Feb. 18, 2022.

(51) Int. Cl. A61K 9/00; A61K 35/30; A61K 48/00

(52) U.S. Cl. CPC A61K 9/0048; A61K 9/0009; A61K 9/0019; A61K 35/30; A61K 48/00

(57) ABSTRACT

The present disclosure concerns methods of administering a therapeutic agent to the subretinal space of the eye of a subject: creating a local retinal detachment in the subretinal space of an eye of the subject; injecting a composition comprising a magnetized therapeutic agent and a volume of a pharmaceutically acceptable carrier; and applying a magnetic force to the eye, wherein the magnetic force adheres the magnetized therapeutic agent to the subretinal space of the eye. Additionally, the method comprises removing at least 50% of the volume of pharmaceutically acceptable carrier from the eye while the magnetic force is applied to the eye.

### RELATED APPLICATIONS

This application claims priority to and the benefit of U.S. provisional patent application 63/311,718, filed Feb. 18, 2022, titled “Methods of Administering Therapeutic Agents to the Subretinal Space,” to Emmetrope Ophthalmics LLC, the entirety of the disclosure is hereby incorporated herein by reference thereto.

### TECHNICAL FIELD

This document relates to methods of administering a therapeutic agent to the subretinal space of the eye in a subject and a method of treating a retinal disorder in a patient.

### BACKGROUND

Although cell therapy for ocular regeneration, such as cell injection for endothelial cell dysfunction and stem cell transplantation for retinal neuroprotection have been studied, these procedures are challenging technically, have varying rates of success, and are not yet commercially available for many types of ocular diseases or disorders, including diseases of the cornea (including but not limited to endothelial dystrophies), diseases of retinal ganglion cells and the optic nerve (including but not limited to glaucoma, ischemic optic neuropathies, other optic neuropathies), and diseases of retinal photoreceptors and retinal pigment epithelium (including but not limited to Leber's congenital amaurosis, retinitis pigmentosa and age-related macular degeneration).

Unlike cell transplantation therapy in other organs, simply injecting therapeutic cells or even other therapeutic agents into the eye generally does not work as they do not remain localized and stick to or become incorporated into the patient's tissue. For example, healthy corneal endothelial cells are inefficiently incorporated in a pre-clinical model of corneal endothelial dysfunction when those cells are injected into the anterior chamber of the eye (e.g., Mimura et al., Invest. Ophthalmol. Vis. Sci. 2005, 46 (10): 3637-44). Similarly, healthy retinal ganglion cells are not incorporated into the correct retinal layer by simple injection into a cavity of the eye. Most current cell therapy technology for the eye lack techniques for controlling the cells' localization in vivo. A stem cell transplantation clinical trial for retinitis pigmentosa uses subretinal injection of hematopoietic stem cells with no mechanism for controlling the localization of the stem cells to the subretinal space and to keep them from floating or migrating away in the fluid of the eye. As another example, corneal endothelial cells injected into the anterior chamber of the eye will simply fall by gravity away from the cornea and not properly attach unless they are co-injected with a molecule that promotes cell adhesion (Kinoshita et al., N Engl J Med. 2018, 378(11): 995-1003). Thus, there remains a need for new methods for targeting cells to specific tissues for therapeutic purposes.

### SUMMARY

In some aspects, the disclosure concerns methods of administering a therapeutic agent to the subretinal space of the eye of a subject; creating a local retinal detachment in the subretinal space of an eye of the subject; injecting a composition comprising a magnetized therapeutic agent and a volume of a pharmaceutically acceptable carrier; and applying a magnetic force to the eye, wherein the magnetic force adheres the magnetized therapeutic agent to the subretinal space of the eye. In some embodiments, the process additionally requires removing at least 10%, 20%, 30%, 40% or 50% the volume of the pharmaceutically acceptable carrier from the eye while the magnetic force is applied to the eye.

In other aspects, the disclosure concerns methods of treating a retinal disorder in a subject, the method comprising: creating a local retinal detachment in the subretinal space of an eye of the subject; injecting a composition comprising a magnetized therapeutic agent and a volume of a pharmaceutically acceptable carrier; applying a magnetic force to the eye, wherein the magnetic force adheres the magnetized therapeutic agent to the subretinal space of the eye. In a particular embodiment, the method further comprises removing at least 10%, 20%, 30%, 40%, 50% or 60% of the volume of the pharmaceutically acceptable carrier from the eye while the magnetic force is applied to the eye. For example, in a exemplary embodiments, 80-99%, the volume of the pharmaceutically acceptable carrier is removed while the magnetic force is applied to the eye.

In certain embodiments the disclosure concerns use of (i) a composition comprising magnetized therapeutic agent and a pharmaceutically acceptable carrier and (ii) a magnetic force to guide and adhere the composition to the subretinal space of the eye to treat a condition or disease, for example, treatment of endothelial dystrophy, glaucoma, ischemic optic neuropathy, myopic degeneration, Leber's congenital amaurosis, retinitis pigmentosa, diabetic macular edema, proliferative diabetic retinopathy, retinopathy of prematurity, macular degeneration, age-related macular degeneration, or combination thereof.

In certain embodiments, the magnetized therapeutic agent comprises a magnetic particle and a therapeutic agent, the magnetic particle is affixed to the therapeutic agent and the therapeutic agent is selected from the group consisting of: a gene therapy agent, an ocular cell, and a therapeutic drug.

In certain methods, the ocular cell selected from the group consisting of: a retinal pigment epithelial cell, a photoreceptor cell, a bipolar cell, a ganglion cell, a horizontal cell, an amacrine cell, a stem cell, a retinal precursor cell, and a stem cell-derived ocular cell.

Some method further comprise stopping application of the magnetic force to the eye once at least 50% the volume of the pharmaceutically acceptable carrier has been removed.

In certain embodiments, the magnetized therapeutic agent is a magnetized ocular cell, the application of the magnetic force to the eye is stopped once the magnetized ocular cell has localized to the Bruch's membrane, or to the retinal pigment epithelium layer, or to the posterior layer of the neural retina.

Some embodiments further comprise injecting a second composition comprising a second magnetized therapeutic agent into the subretinal space of the eye and a volume of a second pharmaceutically acceptable carrier; applying a magnetic force to the eye, wherein the magnetic force adheres the second magnetized therapeutic agent to the subretinal space of the eye; and removing at least 50% the volume of the second pharmaceutically acceptable carrier from the eye while the magnetic force is applied to the eye.

In some embodiments, the second magnetized therapeutic agent is a different magnetized therapeutic agent than the first magnetized therapeutic agent.

In certain embodiments, the first and second magnetized therapeutic agents comprise ocular cells.

In some embodiments, the first magnetized therapeutic agent comprises a retinal pigment epithelial cell and the second magnetized therapeutic agent comprises an ocular cell selected from the group consisting of: photoreceptor cells, bipolar cells, ganglion cells, horizontal cells, and amacrine cells.

In certain embodiments, 80-99% the volume of the second pharmaceutically acceptable carrier is removed while the magnetic force is applied to the eye.

Some methods further comprise stopping application of the magnetic force to the eye once at least 50% the volume of the second pharmaceutically acceptable carrier has been removed.

The local retinal detachment, in some embodiments, may be created by injecting a balanced salt solution into the subretinal space of the eye.

In some methods, the local retinal detachment is created by injecting the composition comprising magnetized therapeutic into the subretinal space of the eye.

Certain embodiments further comprise: removing an amount of vitreous through the pars plana; and creating an incision at the retina at the subretinal space, wherein the composition comprising magnetized ocular cells is injected into the subretinal space of the eye through the incision at the retina.

In some embodiments, the magnetic force pulls the magnetized therapeutic agent towards the subretinal space of the eye.

In certain embodiments, the magnetic force is applied by placing a magnet behind the eye.

In some embodiments, the magnet is placed internally to the subject. In certain embodiments, the magnetic is stitched in place.

In yet other embodiments, the magnet is placed outside the subject.

In some embodiments, the magnetic force is applied by an external 3D magnet.

Certain magnetic particles have a mean diameter of no more than 1 micron, no more than 500 nm, no more than 200 nm, or no more than 50 nm. Some magnetic particles have a diameter of no more than 1 micron, no more than 500 nm, or no more than 200 nm.

Some magnetic particles comprise iron in any ferromagnetic form. Certain magnetic particles have a surface coating. The surface coating can allow the binding of an antibody, antibody fragment, chemical moiety, protein or sugar fragment that binds to therapeutic agent.

The foregoing and other aspects, features, and advantages will be apparent from the DESCRIPTION and DRAWINGS, and from the CLAIMS if any are included.

### BRIEF DESCRIPTION OF THE DRAWINGS

The drawings illustrates certain non-limiting embodiments and implementations.

FIG. 1A illustrates one magnetic surgical apparatus positioning a magnetic body behind the eye of the patient.

FIG. 1B presents one perspective view of a patient eye shown without the context of the surrounding eye socket or other structures and tissue

Accordingly, it is to be understood that the embodiments of the invention herein described are merely illustrative of the application of the principles of the invention. Reference herein to details of the illustrated embodiments is not intended to limit the scope of the claims, which themselves recite those features regarded as essential to the invention.

### DETAILED DESCRIPTION

Detailed aspects and applications of the disclosure are described below in the following drawings and detailed description of the technology. Unless specifically noted, it is intended that the words and phrases in the specification and the claims be given their plain, ordinary, and accustomed meaning to those of ordinary skill in the applicable arts.

In the following description, and for the purposes of explanation, numerous specific details are set forth in order to provide a thorough understanding of the various aspects of the disclosure. It will be understood, however, by those skilled in the relevant arts, that embodiments of the technology disclosed herein may be practiced without these specific details. It should be noted that there are many different and alternative configurations, devices and technologies to which the disclosed technologies may be applied. The full scope of the technology disclosed herein is not limited to the examples that are described below.

The word “exemplary,” “example,” or various forms thereof are used herein to mean serving as an example, instance, or illustration. Any aspect or design described herein as “exemplary” or as an “example” is not necessarily to be construed as preferred or advantageous over other aspects or designs. Furthermore, examples are provided solely for purposes of clarity and understanding and are not meant to limit or restrict the disclosed subject matter or relevant portions of this disclosure in any manner. It is to be appreciated that a myriad of additional or alternate examples of varying scope could have been presented, but have been omitted for purposes of brevity.

When a range of values is expressed, another embodiment includes from the one particular value and/or to the other particular value. Similarly, when values are expressed as approximations, by use of the antecedent “about,” it will be understood that the particular value forms another embodiment. All ranges are inclusive and combinable.

Throughout the description and claims of this specification, the words “comprise” and “contain” and variations of the words, for example “comprising” and “comprises”, mean “including but not limited to”, and are not intended to (and do not) exclude other components.

As required, detailed embodiments of the present disclosure are included herein. It is to be understood that the disclosed embodiments are merely exemplary of the disclosure that may be embodied in various forms. Therefore, specific structural and functional details disclosed herein are not to be interpreted as limits, but merely as a basis for teaching one skilled in the art to employ the present disclosure. The specific examples below will enable the disclosure to be better understood. However, they are given merely by way of guidance and do not imply any limitation.

The present disclosure may be understood more readily by reference to the following detailed description taken in connection with the accompanying figures and examples, which form a part of this disclosure. It is to be understood that this disclosure is not limited to the specific materials, devices, methods, applications, conditions, or parameters described and/or shown herein, and that the terminology used herein is for the purpose of describing particular embodiments by way of example only and is not intended to be limiting of the claimed inventions. The term “plurality”, as used herein, means more than one. When a range of values is expressed, another embodiment includes from the one particular value and/or to the other particular value. Similarly, when values are expressed as approximations, by use of the antecedent “about,” it will be understood that the particular value forms another embodiment. All ranges are inclusive and combinable.

This disclosure, its aspects and implementations, are not limited to the specific material types, components, methods, or other examples disclosed herein. Many additional material types, components, methods, and procedures known in the art are contemplated for use with particular implementations from this disclosure. Accordingly, for example, although particular implementations are disclosed, such implementations and implementing components may comprise any components, models, types, materials, versions, quantities, and/or the like as is known in the art for such systems and implementing components, consistent with the intended operation.

While this disclosure includes embodiments of many different forms, there is shown in the drawings and will herein be described in detail particular embodiments with the understanding that the present disclosure is to be considered as an exemplification of the principles of the disclosed methods and systems, and is not intended to limit the broad aspect of the disclosed concepts to the embodiments illustrated.

All amounts, ratios, and percentages are by weight unless otherwise indicated. The articles “a”, “an”, and “the” each refer to one or more, unless otherwise indicated by the context of the specification. Thus, for example, reference to “a step” includes reference to one or more of such steps.

The disclosure of ranges includes the range itself and also anything subsumed therein, as well as endpoints. For example, disclosure of a range of 2.0 to 4.0 includes not only the range of 2.0 to 4.0, but also 2.1, 2.3, 3.4, 3.5, and 4.0 individually, as well as any other number subsumed in the range. Furthermore, disclosure of a range of, for example, 2.0 to 4.0 includes the subsets of, for example, 2.1 to 3.5, 2.3 to 3.4, 2.6 to 3.7, and 3.8 to 4.0, as well as any other subset subsumed in the range. As used herein “about,” “approximately,” and “substantially” mean within a percent difference of less than or equal to 20%, 10%, 5%, 3%, 2%, or 1%. Similarly, the disclosure of Markush groups includes the entire group and also any individual members and subgroups subsumed therein.

Described herein are a method of administering a therapeutic agent to the subretinal space of the eye in a subject and a method of treating a retinal disorder in a subject. The methods comprise creating a local retinal detachment in the subretinal space of an eye of the subject and injecting a composition comprising a magnetized therapeutic agent and a volume of a pharmaceutically acceptable carrier. A magnetic force is applied to the eye, wherein the magnetic force adheres the magnetized therapeutic agent to the subretinal space of the eye. While the magnetic force is applied to the eye, at least 50% the volume of the pharmaceutically acceptable carrier is removed from the eye. In some implementations, 80-99% the volume of the pharmaceutically acceptable carrier is removed while the magnetic force is applied to the eye. In some embodiments, the methods further comprise stopping application of the magnetic force to the eye once at least 50% the volume of the pharmaceutically acceptable carrier has been removed.

In some aspects, the magnetized therapeutic agent comprises a magnetic particle and a therapeutic agent. The magnetic particle is affixed to the therapeutic agent and the therapeutic agent is selected from the group consisting of: a gene therapy agent, a ocular cell, and a therapeutic drug. In certain implementations, the ocular cell selected from the group consisting of: a retinal pigment epithelial cell, a photoreceptor cell, a bipolar cell, ganglion cell, horizontal cell, amacrine cell, and stem cell-derived ocular cell. In particular embodiments where the magnetized therapeutic agent is a magnetized ocular cell, the application of the magnetic force to the eye is stopped once the magnetized ocular cell has localized to the Bruch's membrane.

The magnetic particle preferably has a mean diameter of no more than 1 micron, no more than 500 nm, no more than 200 nm, or no more than 50 nm. In particular implementations, the magnetic particle has a diameter of no more than 1 micron, no more than 500 nm, or no more than 200 nm. In some aspects, the magnetic particle comprises iron in any ferromagnetic form. In certain embodiments, the magnetic particle has a surface coating. For example, the surface coating allows the binding of an antibody, antibody fragment, protein or sugar fragment that binds to therapeutic agent.

In some aspects, the magnetic force pulls the magnetized therapeutic agent towards the subretinal space of the eye. In certain implementations, the magnetic force is applied by placing a magnet behind the eye. In some aspects, the magnet is placed internally to the subject, for example, the magnetic is stitched in place. In other aspects, the magnet is placed outside the subject. For example, the magnetic force is applied by an external 3D magnet.

In certain implementations of the methods described herein, the method further comprises injecting a second composition comprising a second magnetized therapeutic agent into the subretinal space of the eye and a volume of a second pharmaceutically acceptable carrier and applying a magnetic force to the eye, wherein the magnetic force adheres the second magnetized therapeutic agent to the subretinal space of the eye. While the magnetic force is applied to the eye, at least 50% the volume of the second pharmaceutically acceptable carrier is removed from the eye. In some embodiments, 80-99% the volume of the second pharmaceutically acceptable carrier is removed while the magnetic force is applied to the eye. In some implementations, the method comprise stopping application of the magnetic force to the eye once at least 50% the volume of the second pharmaceutically acceptable carrier has been removed.

In some aspects, the second magnetized therapeutic agent is a different magnetized therapeutic agent than the first magnetized therapeutic agent. In some embodiments, the first and second magnetized therapeutic agents comprise ocular cells. For example, the first magnetized therapeutic agent comprises a retinal pigment epithelial cell and the second magnetized therapeutic agent comprises an ocular cell selected from the group consisting of: photoreceptor cells, bipolar cells, ganglion cells, horizontal cells, and amacrine cells.

In particular implementations of the methods described herein, the local retinal detachment is created by injecting a balanced salt solution into the subretinal space of the eye. in other implementations, the local retinal detachment is created by injecting the composition comprising magnetized therapeutic into the subretinal space of the eye.

In certain embodiments of the methods described herein, the methods further comprise removing an amount of vitreous through the pars plana and creating an incision at the retina at the subretinal space. The composition comprising magnetized ocular cells is injected into the subretinal space of the eye through the incision at the retina.

The term “incision” may also include an injection, a puncture, a retinotomy; alternatively, the subretinal space may be accessed via the supra-choroidal space, with a puncture across the choroid and Bruch's membrane into the subretinal space. In this case, the magnetic force wouldn't be pulling the therapeutic agent “towards the subretinal space” but away from the incision/puncture site. To facilitate removing a portion or all of the carrier, the magnetic force should be pulling the therapeutic away from the incision site.

The term “subject” refers to an animal, for example, a mammal, in particular a human.

The term “ocular disorder” refers to a condition or disease that interferes with the ability of the eye to function properly and/or that negatively affects the visual clarity of the eye.

The term “gene therapy” refers to a therapeutic technique wherein a subject's genes are modified to treat or cure a disease.

The term “pharmaceutically acceptable carrier” refers to a pharmaceutically acceptable material, composition, or vehicle, such as a liquid, diluent, excipient, or solvent involved in carrying or transporting the subject therapeutic agent composition. Each carrier must be “acceptable” in the sense of being compatible with the other ingredients of the formulation and not injurious to a subject to whom it is administered. Nor should an acceptable carrier alter the specific activity of the therapeutic agent.

The term “target tissue” refers to any specific tissue type or location, e.g., within an organ or tissue, to which it is desirable to deliver the therapeutic agent. For example, the therapeutic agent may be delivered to the eye, or a specific region of the eye, e.g., to the cornea, optic nerve, retina, etc.

The term “magnetic nanoparticle” refers to a particle on the nanometer scale, i.e., 1-500 nm, having magnetic properties.

Described herein are methods of delivering of therapeutic agents to the subretinal space of the eye, which also relates to a method of treating a retinal disorder. While many ocular treatments are delivered topically in the form of eyedrops or gels, topical administration does not result in precise and efficient delivery. For delivering therapeutic to the subretinal space, several barriers need to be overcome for effective delivery. Precorneal, dynamic, and static ocular barriers all restrict the delivery of therapeutic agents to ocular tissue. Additionally, therapeutic drug levels are not well maintained in ocular tissue over time. Although injection is a common form of ocular therapeutic delivery that overcomes many of these barriers, repeated eye puncture is associated with endophthalmitis, hemorrhage, retinal detachment and is not tolerated well by patients. For cell-based therapy, the injection of fluid into the subretinal space requires the formation of a bleb until the additional fluid is resorbed over time. However, the presence of the bleb can lead to the formation of scar tissue in the eye, which often can negatively affect eyesight. The described methods of delivering therapeutic agents to the subretinal space of the eye addresses these limitations of existing therapies to treat ocular disorders and diseases. Examples of ocular disorders that may be treated with the method described herein include, but are not limited to, degenerative vitreoretinal diseases, such as retinitis pigmentosa, macular degeneration, and Leber's congenital amaurosis, age-related macular degeneration, diabetic macular edema, proliferative diabetic retinopathy, myopic degeneration, and retinopathy of prematurity.

The methods of delivering therapeutic agents to the subretinal space of the eye described herein comprise creating a local retinal detachment in the subretinal space of an eye of the subject; injecting a composition comprising a magnetized therapeutic agent and a volume of a pharmaceutically acceptable carrier; applying a magnetic force to the eye, wherein the magnetic force adheres the magnetized therapeutic agent to the subretinal space of the eye; and removing at least 50% the volume of the pharmaceutically acceptable carrier from the eye while the magnetic force is applied to the eye. Local retinal detachment may be created by established methods in the art, for example, by injecting a solution into the subretinal space. The solution may be a balanced salt solution or the composition comprising magnetized therapeutic agent. In some implementations, creating the local retinal detachment comprises removing an amount of vitreous through the pars plana and creating an incision at the retina at the subretinal space, wherein the composition comprising magnetized therapeutic agent is injected into the subretinal space of the eye through the incision at the retina.

Suitable pharmaceutical carriers to ocular application are known in the art, for example those described in Remington: The Science and Practice of Pharmacy, 21^{st} Ed. (2005). In some aspects, at least 50% the volume of the pharmaceutically acceptable carrier is removed from the eye at least 1 minute, at least 2 minutes, at least 3 minutes, at least 4 minutes, at least 5 minutes, at least 10 minutes, at least 15 minutes, at least 20 minutes, at least 25 minutes, at least 30 minutes, at least an hour after injection of the composition comprising a magnetized therapeutic agent and a volume of pharmaceutically acceptable carrier. In certain implementations, the pharmaceutically acceptable carrier is removed from the eye no more than 3 hours, no more than 2 hours, or no more than 1 hour after injection of the composition comprising a magnetized therapeutic agent and a volume of pharmaceutically acceptable carrier.

In particular embodiments, at least 50%, at least 60%, at least 70%, at least 80%, at least 90%, at least 91%, at least 92%, at least 93%, at least 94%, at least 95%, at least 96%, at least 97%, at least 98%, or at least 99% volume of the pharmaceutically acceptable carrier is removed from the eye about 5-30 minutes after injection of the composition comprising a magnetized therapeutic agent and a volume of pharmaceutically acceptable carrier. For example, at least 50%, at least 60%, at least 70%, at least 80%, at least 90%, at least 91%, at least 92%, at least 93%, at least 94%, at least 95%, at least 96%, at least 97%, at least 98%, or at least 99% volume of the pharmaceutically acceptable carrier is removed 5-10 minutes after injection, 5-15 minutes after injection, 5-20 minutes after injection, 5-25 minutes after injection, 5-30 minutes after injection, 10-15 minutes after injection, 10-20 minutes after injection, 10-25 minutes after injection, 10-30 minutes after injection, 15-20 minutes after injection, 15-25 minutes after injection, 15-30 minutes after injection, 20-25 minutes after injection, 20-30 minutes after injection, or 25-30 minutes after injection. Thus, in some implementations, between 50-99% volume (for example, 50-60%, 50-70%, 50-80%, 50-90%, 50-95%, 50-99%, 60-70%, 60-80%, 60-90%, 60-95%, 60-99%, 70-80%, 70-90%, 70-95%, 70-99%, 80-90%, 80-95%, 80-99%, 90-95%, 90-99%, 95-99%, or any other range between 50-99%) is removed or evacuated after injection of the composition comprising a magnetized therapeutic agent and a volume of pharmaceutically acceptable carrier. In another aspect, no more than 99% volume of the pharmaceutically acceptable carrier is evacuated after injection of the composition comprising a magnetized therapeutic agent and a volume of pharmaceutically acceptable carrier.

In certain implementations, the magnetic force is applied by a magnet positioned behind the eye of the patient. In other implementations, the magnetic force is applied by a magnet coupled to the patient on the rear of the sclera, behind the retina. The magnet may be coupled to the patient through stitches or any other acceptable manner of coupling. In some aspects, the magnet is positioned behind the eye of the patient in a reversible manner so that the magnet is removable once magnetic force is no longer required.

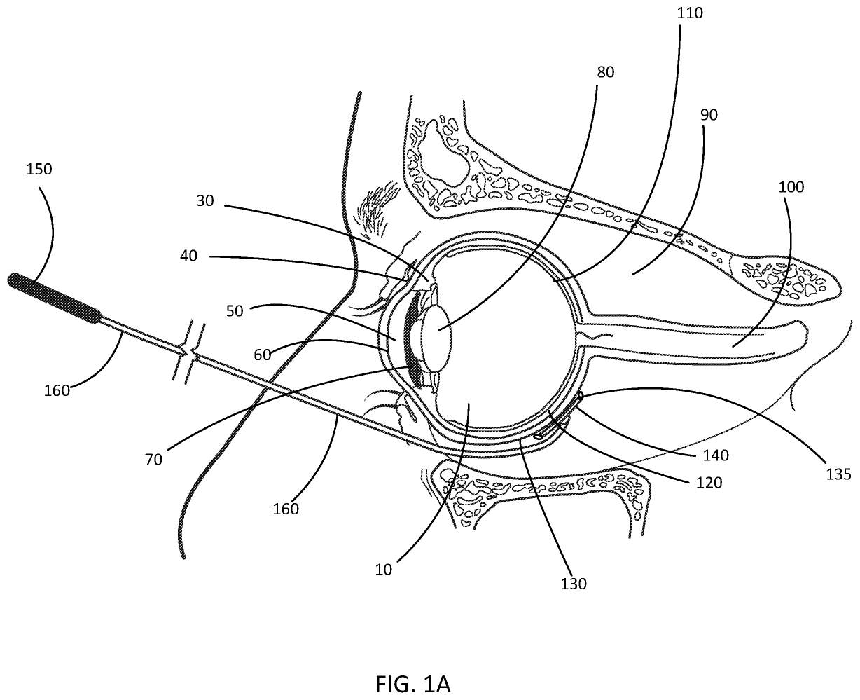

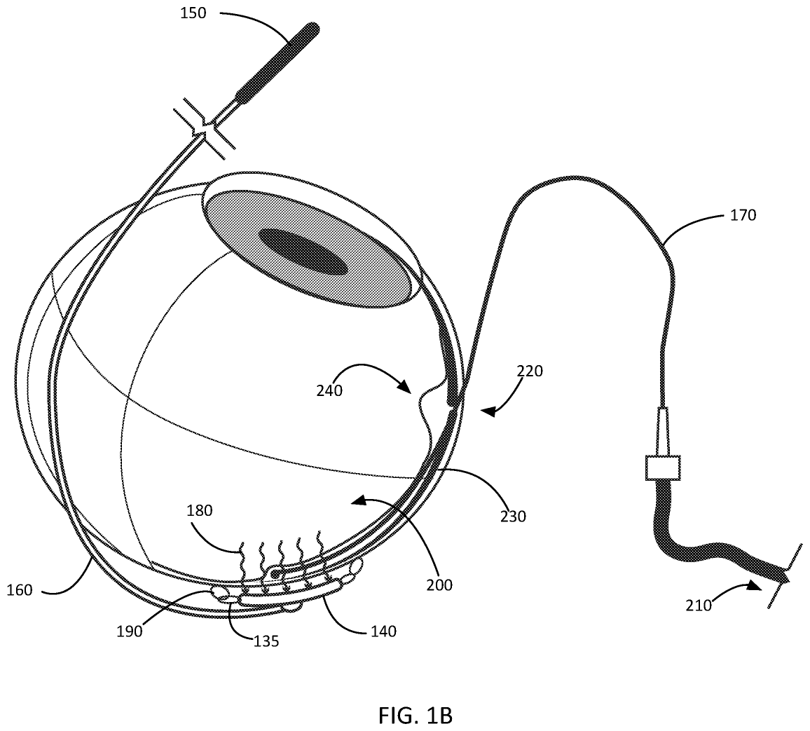

FIG. 1A depicts a magnetic surgical apparatus 135 positioning a magnetic body 140 behind the eye 10 of the patient, where magnets have not previously been placed. The magnetic body 140 is shown being placed by an arm 160 of the magnetic surgical apparatus 135, to which the magnetic body 140 is coupled, the arm 160 of the magnetic surgical apparatus 135 extending through an incision in the skin and around to a desired portion of the patient's eye 10. The arm 160 and the magnetic body 140 may be manipulated and positioned by the handle 150 coupled to the arm 160. FIG. 1A shows additional features of the eye 10 including ciliary body 30, conjunctiva 40, anterior chamber 50, cornea 60, iris 70, lens 80, orbit 90, optic nerve 100, retina 110, choroid 120 and sclera 130.

FIG. 1B is a perspective view of a patient eye 10 shown without the context of the surrounding eye socket or other structures and tissue. Like FIG. 1A, FIG. 1B shows (but with greater magnification) a magnetic surgical apparatus 135 positioning a magnetic body 140 behind or at a rear portion of the patient eye 10, such as over the macula or at an area where retinal cells have degraded, been damaged, or are otherwise not functioning properly and will be replaced or augmented with new magnetic stem cells 200. FIG. 1B further shows a catheter 170 that may be used to place new magnetic stem cells at or near a site of the damaged retina. In some embodiments, the catheter 170 has a connection to a pump 210. FIG. 1B also shows sclerotomy and choroidal fistula 220, subretinal cannulation 230, and Healon lifter retina 240. The magnetic body may be positioned before the placement of the new magnetic cells 200 to assist in the positioning and alignment of the magnetic cells 200. As shown in FIG. 1B, the magnetic body 140 may create a magnetic field 180 that draws the magnetic cells 200 to a desired position. A size, shape, position, and orientation of the magnetic body 140 may be configured to match or approximate a size, shape, position, and orientation of the damaged portion of the eye 10 and the area in which new magnetic cells 200 will be positioned.

Rather than having the magnet being externally applied, such as being affixed in a patch to the surface of the eye 10 external to the eyelid centered over the cornea 60 (so as to push or repel the magnetic cells 200), the magnetic body 140 may be disposed within the patient and proximate the area being treated, such as within the eye socket and behind the eye 10 of the patient (so as to attract or pull the magnetic cells 200 to a desired location).

Where the magnetic body 140 is configured to mateably couple with other structures of tissue, the mateable surface of magnetic body 140 may be correspondingly adjusted. FIG. 1A also shows a number of attachment apparatus 135 coupled to the magnetic body 140. There may be 2, 3, 4, dozens, 1-100, or any desirable number of attachment apparatus, including loops, rings, or openings configured to receive sutures of stitches for coupling the magnetic body 140 to the patient.

In some embodiments, the magnet is left in position for a desirable length of time, during which time the cells may begin to adhere, bond, attach, or grow into place and/or the therapeutic agent may be delivered to and interact with the appropriate tissue. For example, the magnet may be left in place for 5 minutes to 7 days, and in some instances more typically 1 hour to 3 days. The magnetic field 180 would help direct the magnetic therapeutic agent to the desired site in the subretinal space. In a particular embodiment wherein the therapeutic agent is a magnetic nanoparticle-bound cell, the magnetic field 180 directs the cells to the specific site where the nanoparticle-bound cells (such as photoreceptor cells or retinal pigment epithelial cells or stem cells) affix to the vitreal surface or subretinal Bruch's membrane surface of the host/patient, after which time natural cell adhesion would take place, the magnetic field 180 may be removed, and the internal magnet could be removed. In certain implementations, the application of the magnetic force to the eye 10 is stopped once the appropriate volume of the pharmaceutically acceptable carrier has been removed.

The fixed/rare earth magnets, electromagnets, or superconducting magnets may comprise sufficient field density uniformity and magnetic field 180 gradient to direct the cells and hold them in place. In some aspects, upon being positioned in the appropriate location behind the eye 10, the magnet is capable of being turned on and off as magnetic force is required. Specifics of magnetic field 180 strength may vary by need, such that stronger fields/gradients being used when the magnet is required to act at greater distances, across larger areas or surfaces of damaged cells, and weaker fields/gradients may be used when the magnet can be localized closer to the implanted particles and/or target tissues. The magnetic therapeutic agent may be directed to the target tissue and with a first magnetic field of a first strength, with the magnetic field 180 then (or thereabout) being modulated to further refine their movement and shape the tissue. The magnetic field 180 may be modulated to be a second magnetic field of a second strength less than the first strength to merely hold the magnetic therapeutic agent in a desired location until a desired adhesion or therapeutic delivery is achieved, rather than using the stronger first field to move the therapeutic agent.

The magnetic therapeutic agents are therapeutic agents that are magnetized with magnetic nanoparticles. The magnetic nanoparticles have a mean diameter of no larger than about 500 nm, more usually no larger than 200 nm, though preferably no more than 100 nm. Particles that can be used include nanospheres, conjugates, micelles, colloids, aggregates, and complexes comprising ferromagnetic, paramagnetic, or superparamagnetic material, such as iron, nickel, cobalt and alloys thereof, as suitable for in vivo use. For example, the magnetic nanoparticles may comprise iron in any ferromagnetic form, with or without an inert surface coating, with its surface chemically modified to allow the binding of an antibody, or antibody fragment, or protein, or sugar fragment that binds to cells. Persons of skill in the art will appreciate that compounds having excessive toxicity when used according to the method are to be avoided. It is expected that in many applications the particles will be endocytosed and excreted over time, and that small amounts of otherwise toxic particles may accordingly not present a problem when used in the method.

In general, the magnetic nanoparticles will have a diameter of between 5 and 500 nm, more particularly between 40 and 400 nm, most particularly between 40 and 100 nm. Difficulties in using nanoparticles over micron-scale particles include particle aggregation, particle tracking and observation, and the ability to mobilize particles by external magnetic fields 180, all of which are considerably easier when using micron-scale or larger particles. For this reason, previous efforts likely focused on micron-scale particles and ignored the possible advantages of using nanoparticles over micron-scale particles. Advantages include the ability to bind to cell surfaces without stimulating endocytosis; the ability to be shed from cell surfaces, or, when internalized, excreted from cells; and the ability to be excreted from the eye 10 or the body when shed from cells.

Magnetic nanoparticles in various forms are already in use clinically and in research applications without any demonstrated toxicity. For example, superparamagnetic particles containing microcrystalline iron oxide nanoparticles (MION) of diameters of <50 nm have been used as MRI contrast agents. These particles have demonstrated neurologic non-toxicity and axonal transport of ferrous-based agents (Neuwell et al., Neurosurgery. 1994, 34:777-784). Published studies supporting the use of MRI contrast agent Ferridex (Advanced Magnetics and Berlex Laboratories) have found no deleterious effects. Furthermore, magnetically directed drug delivery, using tagged pharmaceuticals in the form of magnetic microspheres and magnetic polymer carriers, has shown success in delivering anti-neoplastic drugs and radio-isotypes to magnetically targeted areas in vivo (Schutt et al., Hybridoma. 1997, 16:109-117; Lubbe et al., J Surg Res. 2001, 95:200-206). In particular embodiments, the magnetic nanoparticles are those described in Miltenyi et al., Cytometry. 1990, 11:231-238. For example, the magnetic nanoparticles are dextran coated and have diameters of 30±20 nm (as determined by electron microscopy) or 65±20 nm (as determined by dynamic light scattering). In certain embodiments, the magnetic nanoparticles are CliniMACS® from Miltenyi Biotec, which have a mean diameter of 50 nm.

In some aspects, the magnetic particles are coated. Coatings to be affixed to the magnetic particles include non-specific binding agents such as inert metals like gold or dextrans or polymers; and/or specific binding agents such as antibodies that are specific to cell-surface antigens. For example, antibodies directed against SSEA-1 bind to many types of stem cells, and nanoparticles coated with anti-SSEA-1 antibodies can be used to convert stem cells into magnetic stem cells. Similarly, many cells express specific surface receptors, and antibodies against these specific receptors conjugated to magnetic nanoparticles bind to these cells such as corneal endothelial cells and create magnetic corneal endothelial cells. Coatings are affixed to the particles by standard methods used broadly in the field (for example, see Schroder et al., J Immunol Methods. 1986, 93:45-53; Douglas et al., Crit Rev Ther Drug Carrier Syst. 1987, 3:233-261; Sestier et al., Electrophoresis. 1998, 19:1220-1226; Perrin et al., J Immunol Methods. 1999, 224:77-87; McCloskey et al., Cytometry. 2000, 40:307-315; Tibbe et al., Cytometry. 2001, 43:31-37).

The magnetic nanoparticles can be endocytosed by a cell or affixed to the surface of the cells by any effective means known to those of skill in the art. For example, the magnetic particle may be affixed to the cell by means of an antibody, e.g., an antibody specific for a surface antigen present on the cell. Surface coatings comprising, for example, anti-L1, anti-trkB, anti-integrin, and cholera toxin subunit B may be placed on the magnetic particles for the purpose of attaching them to retinal ganglion cells. The magnetic particle may also be affixed to a cell using a specific ligand for which a receptor is present on the cell. For example, the magnetic particles can be functionalized for attachment to retinal ganglion cells using brain-derived neurotrophic factor (BDNF). Coated magnetic nanoparticles can be affixed to the outer surface of cells by co-incubation in a general media that affords adequate cell survival during the co-incubation period the media is not generally found to be germane to the affixing process. In general, a balanced salt solution at a physiologic pH around 7.4 will suffice; supplements to the media that enhance cell survival during the process are the topic of other published work specific to the cell types being used and are not germane to this disclosure. Co-incubation time and temperature may depend on the specific cell type being converted into a magnetic cell; for example, binding to retinal ganglion cells using magnetic nanoparticles coated with an anti-trkB antibody occurs maximally after 4 hours at 37° C. but may also be performed at 4° C. overnight. Excess magnetic nanoparticles not bound to cells can be washed away either by spinning the cells down in a centrifuge at a speed that pellets the cells but not the unbound magnetic nanoparticles, or by eluting the magnetic nanoparticle-bound cells away from the unbound cells using a magnetic field 180, or both.

Other means of attaching the magnetic particles to a therapeutic agent include non-specific chemical modifications such as carboxy or amide groups, or coatings of sugars or dextrans, or coatings of polymers such as amino acid polymers like poly-lysine, or coatings of otherwise inert coatings that bind to the therapeutic agent.

In still another aspect, the magnetized therapeutic agent is an ocular cell, for example, a retinal cell (whether retinal muller glial cell or retinal astrocyte or other), a retinal endothelial cell or pericyte, a retinal progenitor cell, a retinal stem cell, an optic nerve glial cell (whether an astrocyte, an oligodendrocyte, a microglial cell, or their precursors), or other stem or progenitor cell capable of differentiating into ocular or optic nerve cell or supporting the survival or growth or normal function of an ocular or optic nerve cell. In some embodiments, the ocular cells are selected from the group consisting of: retinal pigment epithelial cells, photoreceptor cells, bipolar cells, ganglion cells, horizontal calls, amacrine cells, and stem cells. In some aspects, the magnetized ocular cells are cells coated with magnetic nanoparticles. Also provided are normal or genetically modified cell(s) having a magnetic nanoparticle endocytosed, bound, or affixed to its surface covalently or by antibody-antigen linkage.

The cells may be suspended in any pharmaceutically/physiologically acceptable medium or solution, such as for example, isotonic saline solution, culture medium, or transport medium suitable for in vivo delivery to a subject. Additional excipients and carriers may be added as are found suitable by those of skill in the art. Suitable solutions and delivery vehicles are described in Remington: The Science and Practice of Pharmacy, 21^{st} Ed. (2005). For some applications, such as delivery of stem cell-derived cells under the retina for age-related macular degeneration or retinitis pigmentosa, 10³-10⁶ cells will be delivered by injection in a volume of 3-300 μL, but more typically around 10⁴ cells in a volume of 10-100 μL. In other applications, such as delivery of stem cells to enhance retinal ganglion cell survival in diseases like glaucoma or ischemic optic neuropathy, 10³-10⁶ cells will be delivered by injection in a volume of 3-300 μL, but more typically around 10⁵ cells in a volume of 200 μL. When considering delivery of cells meant for carrying toxic compounds to specific tissues, for example in cancer therapeutics, the cell number will have to be carefully titrated against systemic toxicity to the patient.

In some aspects, the method described herein comprises administering a second magnetized therapeutic agent after the first magnetized therapeutic agent is delivered. The second magnetized therapeutic agent can be the same as the first magnetized therapeutic agent, or it can be different from the first magnetized therapeutic agent. Preferably, 50-99% volume of the pharmaceutically acceptable carrier in the composition with the first magnetized therapeutic agent is removed from the eye 10 prior to injecting the composition comprising the second magnetized therapeutic agent and a volume of a second pharmaceutically acceptable carrier.

In an exemplary embodiment, the first magnetized therapeutic agent comprises magnetized ocular cell, and the second magnetized therapeutic agent also comprises magnetized ocular cells. In some aspects, the second magnetized therapeutic agent comprises different types of ocular cells than the first magnetized therapeutic agent, for example, to rebuild the cellular structure of the retina or to rebuild the retinal pigment epithelium. During the administration of the first magnetized therapeutic agent, the magnetic field 180 may be modulated by activating different zones, portions, or areas of the magnet to encourage movement of migrations of the cells to particular areas. Cell migration may be directed in real time while observing the placement of cells, such as when cells include a visual marker and may be observed visually during placement. Cell placement may also be observed or monitored indirectly or in any other suitable way. Once the cells achieve a desired adhesion to the target tissue, the pharmaceutically acceptable carrier is evacuated. The time to desired adhesion may be as soon as 1 minute or 5 minutes after injection, or as long as 3 hours after injection, and one skilled in the art may fine tune the time required for cell adhesion to target tissue to take place and the amount and duration of magnetic force required before the cells have adequately adhered and the pharmaceutically acceptable carrier can be evacuated. After the pharmaceutically acceptable carrier from the first therapeutic agent is evacuated, a second therapeutic agent may be administered through the described method and the magnetic ocular cells can be directed to the desired target tissue through the magnetic force. This method allows for multiple layers or strata of cells may be placed with a delay between layering so as to provide time for adhesion of a first layer, and then a subsequent second (or any number or 1+n) layer of cells may be added. In some instances, a first layer and subsequent layer of cells may comprise a same or similar footprint, while in other instances a footprint of subsequent layers of cells may vary and may include different cell types. In such methods, the number of unadhered cells could be reduced with respect to applications where cells are not drawn and held into place, thereby reducing a number of unadhered cells that would otherwise die and remain free floating away from the desired bonding site.

Other combinations of a first magnetized therapeutic agent and a second magnetized therapeutic agent include, but are not limited to the examples listed below:
First magnetized therapeutic agent comprises an ocular cell and the second magnetized therapeutic agent comprises a therapeutic drug;
First magnetized therapeutic agent comprises an ocular cell and the second magnetized therapeutic agent comprises a gene therapy agent;
First magnetized therapeutic agent comprises a first therapeutic drug and the second magnetized therapeutic agent comprises a second therapeutic drug;
First magnetized therapeutic agent comprises a therapeutic drug and the second magnetized therapeutic agent comprises an ocular cell;
First magnetized therapeutic agent comprises a therapeutic drug and the second magnetized therapeutic agent comprises a gene therapy agent;
First magnetized therapeutic agent comprises a gene therapy agent and the second magnetized therapeutic agent comprises a therapeutic drug;
First magnetized therapeutic agent comprises a gene therapy agent and the second magnetized therapeutic agent comprises an ocular cell; or
First magnetized therapeutic agent comprises a gene therapy agent and the second magnetized therapeutic agent comprises a gene therapy agent.

In yet other embodiments, the method further comprises administration of a third, fourth, fifth, or sixth magnetized therapeutic agent following administration of the first and second magnetized therapeutic agent and evacuation of the respective pharmaceutically acceptable carriers. In such implementations, the application of the magnetic force to the eye 10 is stopped once 50-99% volume of the volume of the pharmaceutically acceptable carrier in the composition of the last administered magnetized therapeutic agent is removed from the eye 10.

It will be understood that the embodiments disclosed are not limited to the specific components disclosed herein, as virtually any components consistent with the intended operation of a method and/or system implementation for such an embodiment may be utilized. Accordingly, for example, although particular component examples may be disclosed, such components may be comprised of any shape, size, style, type, model, version, class, grade, measurement, concentration, material, weight, quantity, and/or the like consistent with the intended purpose, method and/or system of implementation. In places where the description above refers to particular implementations or embodiments, it should be readily apparent that a number of modifications may be made without departing from the scope and/or spirit thereof and that these principles and modifications may be applied to other such embodiments. The presently disclosed embodiments are, therefore, to be considered in all respects as illustrative and not restrictive.

Many additional material types, components, methods, and procedures known in the art are contemplated for use with particular implementations from this disclosure. Accordingly, for example, although particular implementations are disclosed, such implementations and implementing components may comprise any components, models, types, materials, versions, quantities, and/or the like as is known in the art for such systems and implementing components, consistent with the intended operation. Additional implementations are possible and encompassed by this disclosure. Thus, further implementations and embodiments based on the teachings of this disclosure are within the claims.

1. A method of administering a therapeutic agent to the subretinal space of the eye of a subject:
creating a local retinal detachment in the subretinal space of an eye of the subject;
injecting a composition comprising a magnetized therapeutic agent and a volume of a pharmaceutically acceptable carrier; and
applying a magnetic force to the eye, wherein the magnetic force adheres the magnetized therapeutic agent to the subretinal space of the eye.

2. A method of treating a retinal disorder in a subject, the method comprising:
creating a local retinal detachment in the subretinal space of an eye of the subject;
injecting a composition comprising a magnetized therapeutic agent and a volume of a pharmaceutically acceptable carrier; and
applying a magnetic force to the eye, wherein the magnetic force adheres the magnetized therapeutic agent to the subretinal space of the eye.

3. The method of claim 1, wherein the administration to the subject is for treatment of endothelial dystrophy, glaucoma, ischemic optic neuropathy, myopic degeneration, Leber's congenital amaurosis, retinitis pigmentosa, diabetic macular edema, proliferative diabetic retinopathy, retinopathy of prematurity, macular degeneration, or age-related macular degeneration.

4. The method of claim 1, additionally comprising removing at least 50% of the volume of pharmaceutically acceptable carrier from the eye while the magnetic force is applied to the eye.

5. The method of claim 1, wherein 80-99% the volume of the pharmaceutically acceptable carrier is removed while the magnetic force is applied to the eye.

6. The method of claim 1, wherein the magnetized therapeutic agent comprises a magnetic particle and a therapeutic agent, the magnetic particle is affixed to the therapeutic agent and the therapeutic agent is selected from the group consisting of: a gene therapy agent, an ocular cell, and a therapeutic drug.

7. The method or use of claim 6, wherein the ocular cell selected from the group consisting of: a retinal pigment epithelial cell, a photoreceptor cell, a bipolar cell, a ganglion cell, a horizontal cell, an amacrine cell, a stem cell, a retinal precursor cell, and a stem cell-derived ocular cell.

8. The method of claim 1, further comprising stopping application of the magnetic force to the eye once at least 50% the volume of the pharmaceutically acceptable carrier has been removed.

9. The method of claim 1, wherein the magnetized therapeutic agent is a magnetized ocular cell, the application of the magnetic force to the eye is stopped once the magnetized ocular cell has localized to the Bruch's membrane, or to the retinal pigment epithelium layer, or to the posterior layer of the neural retina.

10. The method of claim 1, further comprising injecting a second composition comprising a second magnetized therapeutic agent into the subretinal space of the eye and a volume of a second pharmaceutically acceptable carrier;
applying a magnetic force to the eye, wherein the magnetic force adheres the second magnetized therapeutic agent to the subretinal space of the eye; and
removing at least 50% the volume of the second pharmaceutically acceptable carrier from the eye while the magnetic force is applied to the eye.

11. (canceled)

12. The method of claim 10, wherein the second magnetized therapeutic agent is a different magnetized therapeutic agent than the first magnetized therapeutic agent and/or the first and second magnetized therapeutic agents comprise ocular cells.

13. (canceled)

14. The method or use of claim 13, wherein the first magnetized therapeutic agent comprises a retinal pigment epithelial cell and the second magnetized therapeutic agent comprises an ocular cell selected from the group consisting of: photoreceptor cells, bipolar cells, ganglion cells, horizontal cells, and amacrine cells.

15. The method of claim 10, wherein 80-99% the volume of the second pharmaceutically acceptable carrier is removed while the magnetic force is applied to the eye.

16. The method of claim 10, further comprising stopping application of the magnetic force to the eye once at least 50% the volume of the second pharmaceutically acceptable carrier has been removed.

17. The method of claim 1, wherein the local retinal detachment is created by injecting a balanced salt solution into the subretinal space of the eye.

18. The method of claim 1, wherein the local retinal detachment is created by injecting the composition comprising magnetized therapeutic into the subretinal space of the eye.

19. The method of claim 17, further comprising:
removing an amount of vitreous through the pars plana; and
creating an incision at the retina at the subretinal space, wherein the composition comprising magnetized ocular cells is injected into the subretinal space of the eye through the incision at the retina.

20. The method of claim 1, wherein the magnetic force pulls the magnetized therapeutic agent towards the subretinal space of the eye, and at least one of the following: the magnetic force is applied by placing a magnet behind the eye, the magnetic is stitched in place, and the magnetic force is applied by an external 3D magnet.

21. (canceled)

22. (canceled)

23. (canceled)

24. (canceled)

25. (canceled)

26. The method or use of claim 1, wherein the magnetic particle has a diameter or mean diameter of no more than 1 micron, no more than 500 nm, no more than 200 nm, or no more than 50 nm.

27. (canceled)

28. The method of claim 1, wherein the magnetic particle comprises iron in any ferromagnetic form, wherein the magnetic particle has a surface coating that allows the binding of an antibody, antibody fragment, chemical moiety, protein, or sugar fragment that binds to a therapeutic agent.

29-30. (canceled)
